(19) United States
(12) Patent Application Publication
(10) Pub. No.: US 2026/0193372 A1
(43) Pub. Date: Jul. 9, 2026

(54) METHOD FOR DECELERATING THE DECAY-DISSOCIATION OF C3BBB COMPLEX

(71) Applicant: Hoffmann-La Roche Inc., Little Falls, NJ (US)

(72) Inventors: Sebastian FENN, Achmühle (DE); Alexander FIDLER, Penzberg (DE); Anton JOCHNER, Oberammergau (DE); Eloed KOERTVELY, Riehen (CH); Jean-Luc MARY, Huningue (FR)

(73) Assignee: Hoffmann-La Roche Inc., Little Falls, NJ (US)

(21) Appl. No.: 19/575,901

(22) Filed: Mar. 23, 2026

Related U.S. Application Data
(63) Continuation of application No. PCT/EP2024/076827, filed on Sep. 25, 2024.

(30) Foreign Application Priority Data
Sep. 25, 2023 (EP) 23199374.2

(51) Int. Cl. C07K 16/28; C07K 14/47; C12N 9/64; C40B 30/04; C40B 40/02

(52) U.S. Cl. CPC C07K 16/2896; C07K 14/472; C12N 9/6424; C12Y 304/21043; C40B 30/04; C40B 40/02; C07K 2317/94

(57) ABSTRACT

The present invention relates to a method for stabilizing the C3bBb complex, a rapidly decaying component of the alternative pathway of the complement system, and stabilized C3bBb complexes and uses thereof.

### CROSS REFERENCE TO RELATED APPLICATIONS

This application is a continuation of International Application No. PCT/EP2024/076827, filed Sep. 25, 2024, which claims priority to EP Application Serial No. 23199374.2, filed Sep. 25, 2023, each of which is incorporated herein by reference in its entirety.

### SEQUENCE LISTING

This application contains a Sequence Listing which has been submitted electronically in XML format and is hereby incorporated by reference in its entirety. Said XML copy, created on Mar. 19, 2026, is named “P38829-US-1_Sequence_Listing.xml” and is 28,154 bytes in size.

### FIELD OF THE INVENTION

The present invention relates to a method for stabilizing the C3bBb complex, a rapidly decaying component of the alternative pathway of the complement system, and stabilized C3bBb complexes and uses thereof.

### BACKGROUND OF THE INVENTION

C3 convertase is a serine protease involved in the complement system, the formation of which can be triggered by three pathways: the classical, the lectin and the alternative pathways. The alternative pathway C3 convertase (C3bBb) is formed by binding of C3 fragment (C3b) to Factor B (FB) and subsequent cleavage by plasma serine protease Factor D (FD) under release of a fragment Ba, usually in the presence of magnesium ions. The remaining Bb fragment bound to C3b forms the functional C3 convertase C3bBb. C3bBb is relatively unstable with a half-life of a few minutes making it unsuitable as a research tool, e.g. for antibody screening. It has been described previously that the presence of nickel ions instead of magnesium ions and the presence of properdin leads to a stabilization of C3bBb (Z. Fishelson, H. J. Müller-Eberhard, C3 convertase of human complement: enhanced formation and stability of the enzyme generated with nickel instead of magnesium. J. Immunol. 129, 2603 (1982)). Yet, the half life of C3bBb formed in presence of nickel ions was only two times longer than the half life of C3bBb formed in presence of magnesium ions which is still unsuitably short for extended research use.

Genetic variants of FB and their effect on clinical pathology of atypical hemolytic uremic syndrome (aHUS) have been described before (J Am Soc Nephrol 25:2053-2065, 2014).

Therefore there is still a need for the provision of stabilized C3bBb, e.g. for the screening of antibodies.

### SUMMARY OF THE INVENTION

The present invention relates to a method for providing antibodies specifically binding to C3bBb complex, comprising:
a) providing a C3bBb complex using recombinant human factor B (FB) comprising a D279G mutation, and
b) screening and selecting an antibody that specifically binds to the C3bBb complex provided in step a).

The invention further relates to a method of decelerating the decay-dissociation of the C3bBb complex, including providing C3bBb complex using recombinant FB comprising a D279G mutation.

Also, the invention relates to a method of providing a stabilized C3bBb complex, comprising contacting human Complement C3 beta chain (C3b) with recombinant human FB in aqueous solution, under subsequent addition of human Factor D (FD), and optionally subsequent purification of C3bBb, characterized in that the recombinant human FB comprises a D279G mutation.

One embodiment of the invention relates to a method of the invention wherein the recombinant FB further comprises a K350N and/or a M458I mutation.

Another embodiment of the invention relates to a method of the invention wherein the C3bBb complex is provided by contacting C3b, FB and FD in aqueous solution comprising nickel ions.

Another aspect of the invention is a recombinant human Factor B protein according to SEQ ID NO: 02. Another aspect of the invention is a recombinant human Factor B protein according to SEQ ID NO: 03.

Yet another aspect of the invention is a stabilized C3bBb complex formed from a recombinant Factor B precursor, wherein the Factor B precursor is a recombinant human Factor B protein according to the invention.

Another aspect of the invention is a stabilized C3bBb complex comprising a Bb subunit of a recombinant human FB protein according to the invention.

In one embodiment the recombinant FB of the invention is used for providing a C3bBb complex, particularly for use in screening molecules specifically binding to the C3bBb complex.

According to the invention the half life of C3bBb may be increased up to several orders of magnitude thus allowing its application as a research tool, e.g. for the screening of antibodies.

### DESCRIPTION OF THE FIGURES

FIG. 1: Annotated version of the amino acid sequence of human Factor B (wt, SEQ ID NO: 17), signal peptide is highlighted in bold letters, the Bb subunit is underlined and amino acid positions 279, 350 and 458 are highlighted with a black background.

FIG. 2: Assembly and decay of C3bBb formed in presence of magnesium ions as described in Example 1.

FIG. 3: Assembly and decay of C3bBb formed in presence of nickel ions as described in Example 1.

### DETAILED DESCRIPTION OF THE INVENTION

### 1. Definitions

The components of the innate immune system, i.e. the different elements from the classical and alternative pathways are referred to herein according to their standard meaning in the art and the termini used herein include recombinant variants of the respective natural components of the innate immune system that maintain functionality and may include variants comprising tags and/or labels.

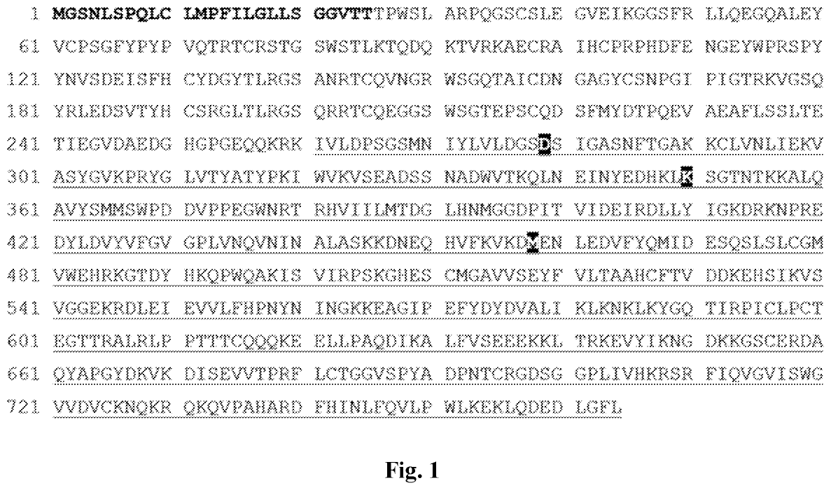

In brief, the term “C3bBb” as used herein refers to the alternative pathway C3 convertase that is formed by binding of C3 fragment (“C3b”) to Factor B (“FB”) and subsequent cleavage by plasma serine protease Factor D (“FD”) under release of a fragment Ba. The term “C3b” as used herein refers to the larger of two elements resulting from the alternative pathway cleavage of complement component 3 (C3), the other one being C3a. The term “C3 convertase” refers to the serine proteases of the complement system and may refer to the alternative pathway C3 convertase (C3bBb) or the classical and lectin pathway C3 convertase (C4bC2b). The term “human Factor B” or “FB” refers to complement factor B encoded in humans by the CFB gene. The term “human Factor D” refers to complement factor D encoded in humans by the CFD gene. The term “C5 convertase” as used herein refers to the alternative pathway C5 convertase (C3bBbC3b). An annotated version of the amino acid sequence of human Factor B (SEQ ID NO: 17) is indicated in FIG. 1. Signal peptide and Bb subunit are highlighted, as are the amino acid positions 279, 350 and 458.

The term “mutation” as referred to herein in connection with amino acid replacements refers to a change from one amino acid to a different amino acid in a protein due to a mutation in the corresponding DNA sequence.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “antibody screening” refers to techniques used to identify antibodies specifically binding to a target antigen. Various techniques for identifying antibodies are known in the art, such as administration of an immunogenic target antigen to a transgenic animal, hybridoma-based methods, or isolating variable domain sequences selected from phage-display libraries. The term “phage library panning” refers to an affinity selection technique used to identify phage display variants with desired binding properties towards a target antigen.

### 2. Detailed Description of the Embodiments of the Invention

One aspect of the invention is a method for providing antibodies specifically binding to C3bBb complex, comprising:
a) providing a C3bBb complex using recombinant human factor B (FB) comprising a D279G mutation, and
b) screening and selecting an antibody that specifically binds to the C3bBb complex provided in step a).

Another aspect of the invention is a method of decelerating the decay-dissociation of the C3bBb complex, including providing C3bBb complex using recombinant FB comprising a D279G mutation.

In one embodiment the recombinant FB further comprises a K350N and/or a M458I mutation. In one embodiment the recombinant FB comprises D279G, K350N and M458I mutations.

It has been demonstrated that by introducing said mutations into FB, the half-life of C3bBb upon formation may be prominently increased, thus resulting in a stabilized C3bBb (Example 1).

In one embodiment, the C3bBb complex provided in a method of the invention is formed by contacting human Complement C3 fragment (C3b) with recombinant human FB comprising a D279G mutation in aqueous solution, under subsequent addition of human Factor D (FD) and subsequent purification of C3bBb. In one embodiment human FB has an amino acid sequence of SED ID NO: 1. In another embodiment, human FB comprising a D279G mutation has an amino acid sequence of SEQ ID NO: 2. In another embodiment, human FB comprising D279G, K350N and M458I mutations has an amino acid sequence of SEQ ID NO: 3. In one embodiment, human Factor D has an amino acid sequence of SEQ ID NO: 4. In one embodiment, human C3b has an amino acid sequence of SEQ ID NO: 5.

In one embodiment, C3b used for providing the C3bBb is conjugated to a label. In one embodiment, C3b used for providing the C3bBb is biotinylated.

In one embodiment, the Bb subunit of the FB used for providing the C3bBb is conjugated to a label. In one embodiment, the Bb subunit of the FB used for providing the C3bBb is biotinylated.

In one embodiment, the C3b and FB used for providing the C3bBb are used in equimolar ratio. In another embodiment the C3bBb is formed using C3b, FB and FD are used in a ratio of 1:1:0.02.

Usually, formation of C3bBb is performed in the presence of magnesium ions. Thus, in one embodiment of the invention the C3bBb complex is provided by contacting C3b, FB and FD in aqueous solution comprising magnesium ions, preferably Mg2+ ions, more preferably MgCl2.

The presence of nickel ions during formation of C3bBb is known to result in an increase of the half-life of the so-formed C3bBb. Hence, in another embodiment, the C3bBb complex is provided by contacting C3b, FB and FD in aqueous solution comprising nickel ions, preferably Ni2+ ions, more preferably NiSO4.

In one preferred embodiment, the C3bBb complex is provided by contacting C3b, recombinant FB comprising D279G, K350N and M458I mutations and FD in aqueous solution comprising nickel ions, preferably Ni2+ ions, more preferably NiSO4.

In another embodiment, the C3bBb is provided by contacting C3b, FB and FD in aqueous solution comprising nickel ions, wherein the aqueous solution is free of magnesium ions.

In one embodiment, the C3bBb provided by contacting C3b, FB and FD is purified, preferably by size exclusion chromatography.

With a method of the invention a stabilized C3bBb complex is formed that comprises a C3b subunit and the Bb subunit of the recombinant FB having the D279G mutation and optionally the further mutations K350N and/or a M458I.

Hence, a further aspect of the invention is a C3bBb complex comprising a Bb subunit of a recombinant human FB with a D279G mutation. In one embodiment the C3bBb complex comprises the Bb subunit of the FB of SEQ ID NO: 2. In one embodiment the C3bBb complex comprises the Bb subunit of a FB protein of SEQ ID NO: 17, wherein amino acid D at position 279 was replaced by G.

Another aspect of the invention is a C3bBb complex comprising a Bb subunit of a recombinant human FB with a D279G mutation further comprising a K350N and/or a M458I mutation. In one embodiment, the C3bBb complex of the invention comprises a Bb subunit of a recombinant human FB with D279G, K350N and M458I mutations. In one embodiment the C3bBb complex comprises the Bb subunit of the FB of SEQ ID NO: 3. In one embodiment the C3bBb complex comprises the Bb subunit of a FB protein of SEQ ID NO: 17, wherein amino acid D at position 279 was replaced by G, amino acid K at position 350 was replaced by N and amino acid M at position 458 was replaced by I.

In one embodiment, the C3bBb complex of the invention comprises a C3b subunit of SEQ ID NO: 5.

The C3bBb according to the invention or provided by a method of the invention, preferably the purified C3bBb, may be used for therapeutic purposes or diagnostic purposes or as a research tool. Thus, another aspect of the invention is the use of C3bBb comprising a Bb subunit of a FB comprising a D279G mutation for therapy or for diagnostic purposes or as a research tool.

In one embodiment, the C3bBb according to the invention is used as a target for screening of antigen binding molecules specifically binding to C3bBb.

Thus, in one embodiment of the method for screening antibodies specifically binding to C3bBb complex according to the invention, the C3bBb complex provided in step a) is used as a target for screening of an antibody that specifically binds to said C3bBb complex.

In one preferred embodiment, the screening is performed by phage library panning.

Another object of the invention is a recombinant human Factor B protein comprising a D279G mutation. In one embodiment, the recombinant human Factor B protein comprising a D279G mutation according to the invention comprises SEQ ID NO: 02.

A further object of the invention is a recombinant human Factor B protein comprising D279G, K350N and M458I mutations. In one embodiment, the recombinant human Factor B protein comprising D279G, K350N and M458I mutations comprises SEQ ID NO: 03.

Both recombinant FB may be used to provide a stabilized C3bBb, as described above. Thus, another aspect of the invention is the recombinant FB of the invention for use in providing a C3bBb complex. An even further aspect of the invention is the recombinant FB of the invention for use in providing a C3bBb complex for use in screening molecules, particularly antibodies, specifically binding to the C3bBb complex.

Another aspect of the invention is the use of the FB of the invention in a method of decelerating the decay-dissociation of the C3bBb complex, preferably a method of the invention.

Another aspect of the invention is the use of the FB of the invention in a method for providing antibodies specifically binding to C3bBb complex, preferably a method of the invention.

As described above, the inventors have shown that by introduction of specific mutations in the Bb subunit of human Factor B a C3bBb complex with a prominently enhanced stability can be formed. The stability and purity of the formed C3bBb may be further improved by providing the C3bBb from its precursors in the presence of nickel ions instead of magnesium ions.

### 3. List of Specific Embodiments of the Invention

In the following, specific embodiments of the invention are listed.
1. A method for providing antibodies specifically binding to C3bBb complex, comprising:
   a) providing a C3bBb complex using recombinant human factor B (FB) comprising a D279G mutation, and
   b) screening and selecting an antibody that specifically binds to the C3bBb complex provided in step a).
2. A method of decelerating the decay-dissociation of the C3bBb complex, including providing C3bBb complex using recombinant FB comprising a D279G mutation.
3. A method of providing a stabilized C3bBb complex, comprising contacting human Complement C3 beta chain (C3b) with recombinant human FB in aqueous solution, under subsequent addition of human Factor D (FD), and optionally subsequent purification of C3bBb, characterized in that the recombinant human FB comprises a D279G mutation.
4. The method of one of embodiments 1 to 3, wherein the recombinant FB further comprises a K350N and/or a M458I mutation.
5. The method of one of embodiments 1 to 3, wherein the recombinant FB further comprises a K350N and a M458I mutation.
6. The method according to any one of the preceding embodiments, wherein the C3bBb complex is formed by contacting human recombinant Complement C3 beta chain (C3b) with recombinant human FB comprising a D279G mutation in aqueous solution, under subsequent addition of recombinant human Factor D (FD) and subsequent purification of C3bBb.
7. The method according to embodiment 6, wherein the C3b is conjugated to a label.
8. The method according to embodiment 6 or 7, wherein the C3b is biotinylated.
9. The method according to any one of embodiments 6 to 8, wherein C3b and FB are used in equimolar ratio.
10. The method according to any one of embodiments 6 to 9, wherein C3b, FB and FD are used in a ratio of 1:1:0.02.
11. The method according to any one of the preceding embodiments, wherein the C3bBb complex is provided by contacting C3b, FB and FD in aqueous solution comprising magnesium ions, preferably Mg²⁺ ions, more preferably MgCl₂.
12. The method according to any one of embodiments 1 to 10, wherein the C3bBb complex is provided by contacting C3b, FB and FD in aqueous solution comprising nickel ions, preferably Ni²⁺ ions, more preferably NiCl₂ or NiSO₄, particularly preferably NiSO4.
13. The method according to embodiment 12, wherein the C3bBb is provided by contacting C3b, FB and FD in aqueous solution comprising nickel ions, wherein the aqueous solution is free of magnesium ions.
14. The method according to any one of the preceding embodiments, wherein the C3bBb complex is purified by size exclusion chromatography.
15. The method for screening antibodies specifically binding to C3bBb complex, according to embodiments 1 and 4 to 14, wherein the C3bBb complex provided in step a) is used as a target for screening of an antibody that specifically binds to said C3bBb complex.
16. The method according to embodiment 15, wherein the screening is performed by phage library panning.
17. A recombinant human Factor B protein comprising a D279G mutation.
18. A recombinant human Factor B protein comprising D279G, K350N and/or M458I mutations.
19. A recombinant human Factor B protein according to SEQ ID NO: 02.
20. A recombinant human Factor B protein according to SEQ ID NO: 03.
21. A stabilized C3bBb complex comprising a Bb subunit of a recombinant human FB with a D279G mutation, preferably a recombinant human Factor B protein according to SEQ ID NO: 02.
22. A stabilized C3bBb complex comprising a Bb subunit of a recombinant human FB with a D279G mutation further comprising a K350N and/or a M458I mutation.
23. A stabilized C3bBb complex comprising a Bb subunit of a recombinant human FB with a D279G mutation further comprising a K350N and a M458I mutation, preferably recombinant human Factor B protein according to SEQ ID NO: 03.
24. A stabilized C3bBb complex formed from a recombinant Factor B precursor, wherein the Factor B precursor is a recombinant human Factor B protein according to one of embodiments 17 to 20.
25. The recombinant human Factor B protein according to one of embodiments 17 to 20 or the stabilized C3bBb complex of one of embodiment 21 to 23 for therapeutic or diagnostic application.
26. The recombinant human Factor B protein according to one of embodiments 17 to 20 for use in providing a C3bBb complex.
27. The recombinant human Factor B protein according to one of embodiments 17 to 20 for use in providing a C3bBb complex for use in screening molecules, particularly antibodies, specifically binding to the C3bBb complex.
28. The recombinant human Factor B protein according to one of embodiments 17 to 20 for use in a method according to any one of embodiments 1 to 16.
29. An antibody specifically binding to C3bBb complex obtained by a method according to any one of embodiments 1 to 16.

### Description of the Amino Acid Sequences

| | |
|---|---|
| SEQ ID NO: 1 | human FactorB |
| | wt (FB) with His Tag and |
| | Precission site |
| | HHHHHHGSLEVLFQGPGGTPWSLARPQGSCSLEGVEIKGGSFRL |
| | LQEGQALEYVCPSGFYPYPVQTRTCRSTGSWSTLKTQDQKTVRK |
| | AECRAIHCPRPHDFENGEYWPRSPYYNVSDEISFHCYDGYTLRG |
| | SANRTCQVNGRWSGQTAICDNGAGYCSNPGIPIGTRKVGSQYRL |
| | EDSVTYHCSRGLTLRGSQRRTCQEGGSWSGTEPSCQDSFMYDTP |
| | QEVAEAFLSSLTETIEGVDAEDGHGPGEQQKRKIVLDPSGSMNI |
| | YLVLDGSDSIGASNETGAKKCLVNLIEKVASYGVKPRYGLVTYA |
| | TYPKIWVKVSEADSSNADWVTKQLNEINYEDHKLKSGTNTKKAL |
| | QAVYSMMSWPDDVPPEGWNRTRHVIILMTDGLHNMGGDPITVID |
| | EIRDLLYIGKDRKNPREDYLDVYVFGVGPLVNQVNINALASKKD |
| | NEQHVFKVKDMENLEDVFYQMIDESQSLSLCGMVWEHRKGTDYH |
| | KQPWQAKISVIRPSKGHESCMGAVVSEYFVLTAAHCFTVDDKEH |
| | SIKVSVGGEKRDLEIEVVLFHPNYNINGKKEAGIPEFYDYDVAL |
| | IKLKNKLKYGQTIRPICLPCTEGTTRALRLPPTTTCQQQKEELL |
| | PAQDIKALFVSEEEKKLTRKEVYIKNGDKKGSCERDAQYAPGYD |
| | KVKDISEVVTPRFLCTGGVSPYADPNTCRGDSGGPLIVHKRSRF |
| | IQVGVISWGVVDVCKNQKRQKQVPAHARDFHINLFQVLPWLKEK |
| | LQDEDLGFL |
| SEQ ID NO: 2 | human FB with |
| | D279G with His Tag and |
| | Precission site |
| | HHHHHHGSLEVLFQGPGGTPWSLARPQGSCSLEGVEIKGGSFRL |
| | LQEGQALEYVCPSGFYPYPVQTRTCRSTGSWSTLKTQDQKTVRK |
| | AECRAIHCPRPHDFENGEYWPRSPYYNVSDEISFHCYDGYTLRG |
| | SANRTCQVNGRWSGQTAICDNGAGYCSNPGIPIGTRKVGSQYRL |
| | EDSVTYHCSRGLILRGSQRRTCQEGGSWSGTEPSCQDSFMYDTP |
| | QEVAEAFLSSLTETIEGVDAEDGHGPGEQQKRKIVLDPSGSMNI |
| | YLVLDGSGSIGASNETGAKKCLVNLIEKVASYGVKPRYGLVTYA |
| | TYPKIWVKVSEADSSNADWVTKQLNEINYEDHKLKSGTNTKKAL |
| | QAVYSMMSWPDDVPPEGWNRTRHVIILMTDGLHNMGGDPITVID |
| | EIRDLLYIGKDRKNPREDYLDVYVFGVGPLVNQVNINALASKKD |
| | NEQHVFKVKDMENLEDVFYQMIDESQSLSLCGMVWEHRKGTDYH |
| | KQPWQAKISVIRPSKGHESCMGAVVSEYFVLTAAHCFTVDDKEH |
| | SIKVSVGGEKRDLEIEVVLFHPNYNINGKKEAGIPEFYDYDVAL |
| | IKLKNKLKYGQTIRPICLPCTEGTTRALRLPPTTTCQQQKEELL |
| | PAQDIKALFVSEEEKKLTRKEVYIKNGDKKGSCERDAQYAPGYD |
| | KVKDISEVVTPRFLCTGGVSPYADPNTCRGDSGGPLIVHKRSRF |
| | IQVGVISWGVVDVCKNQKRQKQVPAHARDFHINLFQVLPWLKEK |
| | LQDEDLGFL |
| SEQ ID NO: 3 | human FB with D279G, |
| | K350N and M458I with His Tag |
| | and Precission site |
| | HHHHHHGSLEVLFQGPGGTPWSLARPQGSCSLEGVEIKGGSFRL |
| | LQEGQALEYVCPSGFYPYPVQTRTCRSTGSWSTLKTQDQKTVRK |
| | AECRAIHCPRPHDFENGEYWPRSPYYNVSDEISFHCYDGYTLRG |
| | SANRTCQVNGRWSGQTAICDNGAGYCSNPGIPIGTRKVGSQYRL |
| | EDSVTYHCSRGLTLRGSQRRTCQEGGSWSGTEPSCQDSFMYDTP |
| | QEVAEAFLSSLTETIEGVDAEDGHGPGEQQKRKIVLDPSGSMNI |
| | YLVLDGSGSIGASNFTGAKKCLVNLIEKVASYGVKPRYGLVTYA |
| | TYPKIWVKVSEADSSNADWVTKQLNEINYEDHKLNSGTNTKKAL |
| | QAVYSMMSWPDDVPPEGWNRTRHVIILMTDGLHNMGGDPITVID |
| | EIRDLLYIGKDRKNPREDYLDVYVFGVGPLVNQVNINALASKKD |
| | NEQHVFKVKDIENLEDVFYQMIDESQSLSLCGMVWEHRKGTDYH |
| | KQPWQAKISVIRPSKGHESCMGAVVSEYFVLTAAHCFTVDDKEH |
| | SIKVSVGGEKRDLEIEVVLFHPNYNINGKKEAGIPEFYDYDVAL |
| | IKLKNKLKYGQTIRPICLPCTEGTTRALRLPPTTTCQQQKEELL |
| | PAQDIKALFVSEEEKKLTRKEVYIKNGDKKGSCERDAQYAPGYD |
| | KVKDISEVVTPRFLCTGGVSPYADPNTCRGDSGGPLIVHKRSRF |
| | IQVGVISWGVVDVCKNQKRQKQVPAHARDFHINLFQVLPWLKEK |
| | LQDEDLGFL |
| SEQ ID NO: 4 | Complement Factor D |
| | ILGGREAEAHARPYMASVQLNGAHLCGGVLVAEQWVLSAAHCLE |
| | DAADGKVQVLLGAHSLSQPEPSKRLYDVLRAVPHPDSQPDTIDH |
| | DLLLLQLSEKATLGPAVRPLPWQRVDRDVAPGTLCDVAGWGIVN |
| | HAGRRPDSLQHVLLPVLDRATCNRRTHHDGAITERLMCAESNRR |
| | DSCKGDSGGPLVCGGVLEGVVTSGSRVCGNRKKPGIYTRVASYA |
| | AWIDSVL |
| SEQ ID NO: 5 | Complement C3 |
| | beta chain (C3b) |
| | SPMYSIITPNILRLESEETMVLEAHDAQGDVPVTVTVHDFPGKK |
| | LVLSSEKTVLTPATNHMGNVTFTIPANREFKSEKGRNKFVTVQA |
| | TFGTQVVEKVVLVSLQSGYLFIQTDKTIYTPGSTVLYRIFTVNH |
| | KLLPVGRTVMVNIENPEGIPVKQDSLSSQNQLGVLPLSWDIPEL |
| | VNMGQWKIRAYYENSPQQVESTEFEVKEYVLPSFEVIVEPTEKF |
| | YYIYNEKGLEVTITARFLYGKKVEGTAFVIFGIQDGEQRISLPE |
| | SLKRIPIEDGSGEVVLSRKVLLDGVQNPRAEDLVGKSLYVSATV |
| | ILHSGSDMVQAERSGIPIVTSPYQIHFTKTPKYFKPGMPFDLMV |
| | FVTNPDGSPAYRVPVAVQGEDTVQSLTQGDGVAKLSINTHPSQK |
| | PLSITVRTKKQELSEAEQATRTMQALPYSTVGNSNNYLHLSVLR |
| | TELRPGETLNVNFLLRMDRAHEAKIRYYTYLIMNKGRLLKAGRQ |
| | VREPGQDLVVLPLSITTDFIPSFRLVAYYTLIGASGQREVVADS |
| | VWVDVKDSCVGSLVVKSGQSEDRQPVPGQQMTLKIEGDHGARVV |
| | LVAVDKGVFVLNKKNKLTQSKIWDVVEKADIGCTPGSGKDYAGV |
| | FSDAGLTFTSSSGQQTAQRAELQCPQPAARRRRSVQLTEKRMDK |
| | VGKYPKELRKCCEDGMRENPMRFSCQRRTRFISLGEACKKVELD |
| | CCNYITELRRQHARASHLGLARSNLDEDIIAEENIVSRSEFPES |
| | WLWNVEDLKEPPKNGISTKLMNIFLKDSITTWEILAVSMSDKKG |
| | ICVADPFEVTVMQDFFIDLRLPYSVVRNEQVEIRAVLYNYRQNQ |
| | ELKVRVELLHNPAFCSLATTKRRHQQTVTIPPKSSLSVPYVIVP |
| | LKTGLQEVEVKAAVYHHFISDGVRKSLKVVPEGIRMNKTVAVRT |
| | LDPERLGREGVQKEDIPPADLSDQVPDTESETRILLQGTPVAQM |
| | TEDAVDAERLKHLIVTPSGCGEQNMIGMTPTVIAVHYLDETEQW |
| | EKFGLEKRQGALELIKKGYTQQLAFRQPSSAFAAFVKRAPSTWL |
| | TAYVVKVESLAVNLIAIDSQVLCGAVKWLILEKQKPDGVFQEDA |
| | PVIHQEMIGGLRNNNEKDMALTAFVLISLQEAKDICEEQVNSLP |
| | GSITKAGDFLEANYMNLQRSYTVAIAGYALAQMGRLKGPLLNKF |
| | LTTAKDKNRWEDPGKQLYNVEATSYALLALLQLKDFDFVPPVVR |
| | WLNEQRYYGGGYGSTQATFMVFQALAQYQKDAPDHQELNLDVSL |
| | QLPSRSSKITHRIHWESASLLRSEETKENEGFTVTAEGKGQGTL |
| | SVVTMYHAKAKDQLTCNKFDLKVTIKPAPETEKRPQDAKNTMIL |
| | EICTRYRGDQDATMSILDISMMTGFAPDTDDLKQLANGVDRYIS |
| | KYELDKAFSDRNTLIIYLDKVSHSEDDCLAFKVHQYENVELIQP |
| | GAVKVYAYYNLEESCTRFYHPEKEDGKLNKLCRDELCRCAEENC |
| | FIQKSDDKVTLEERLDKACEPGVDYVYKTRLVKVQLSNDEDEYI |
| | MAIEQTIKSGSDEVQVGQQRTFISPIKCREALKLEEKKHYLMWG |
| | LSSDFWGEKPNLSYIIGKDTWVEHWPEEDECQDEENQKQCQDLG |
| | AFTESMVVFGCPN |
| SEQ ID NO: 6 | C3bBb.P045.072 VH |
| | KLQLVESGGGLVKPGGSLRLSCAASGRRFGVQDMSWVRQAPGKG |
| | LEWVGSISPKGDHKYLNTKFIGRFTISRDNSKNTLYLQMNSLRA |
| | EDTAVYYCARDWGYFDQWGQGTLVTVSS |
| SEQ ID NO: 7 | C3bBb.P045.072 VL |
| | AIQMTQSPSSLSASVGDRVTITCHGSYWLSSEVAWYQQKPGKAP |
| | KLLIYDGNGIIGNVPSRESGSGSHEDYTLTISSLQPEDFATYYC |
| | QQYRYHPYTFGHGTKVEIK |
| SEQ ID NO: 8 | C3bBb.P048.061 VH |
| | GQLVESGGGLVKPGGSLRLSCKASGFYWGPGAMSWVRQAPGKGL |
| | EWVGSISPKGGSTDINPKIGGRFTISRDNNQDTLYLQMNSLRAE |
| | DTAVYYCARDIGFFDTWGQGTLVTVSS |
| SEQ ID NO: 9 | C3bBb.P048.061 VL |
| | AIQMTQSPSSLSASVGDRVTITCQGSWLISNYLAWYQQKPGKAP |
| | KLLIFDAKYRAPEVPSRFSGSGSGTDFTLTISSLQPEDFATYYC |
| | QQYGGRLLTFGQGTKVEIK |
| SEQ ID NO: 10 | C3bBb.P048.127 VH |
| | GQLVESGGGLVKPGGSLRLSCKASGFYWGPGAMSWVRQAPGKGL |
| | EWVGSISPKGGSTEYNTKVYGRFTISRDNNQDTLYLQMNSLRAE |
| | DTAVYYCARDIGFFDTWGQGTLVTVSS |
| SEQ ID NO: 11 | C3bBb.P048.127 VL |
| | HQMTQSPSSLSASVGDRVTITCDGEWDISNYLAWYQQKPGKAPK |
| | LLIFDAKYRAPEVPSRFSGSGSGRDFTLTISSLQPEDFATYYCQ |
| | QYNRMYFTFGQGTKVEIK |
| SEQ ID NO: 12 | C3bBb.P048.241 VH |
| | GQLVESGGGLVKPGGSLRLSCKASGFYWGPGAMSWVRQAPGKGL |
| | EWVGSISPKGGSAWYNNNFTGRFTISRDNNQDTLYLQMNSLRAE |
| | DTAVYYCARDIGFFDTWGQGTLVTVSS |
| SEQ ID NO: 13 | C3bBb.P048.241 VL |
| | HINQMTQSPSSLSASVGDRVTITCQAKWVISNYLAWYQQKPGKA |
| | PKLLIFDAKYRAPEVPSRFSGSGSDGTFTLTISSLQPEDFATYY |
| | CQQYDWWPYTFGQGTKVEIK |
| SEQ ID NO: 14 | C3bBb.P055.015 VH |
| | GQLVESGGGLVKPGGSLRLSCKASGFYWGPGAMSWVRQAPGKGL |
| | EWVGSISPKGGSAEYASKVYGRFTISRDNNQDTLYLQMNSLRAE |
| | DTAVYYCARDIGFFDTWGQGTLVTVSS |
| SEQ ID NO: 15 | C3bBb.P055.015 VL |
| | DYQMTQSPSSLSASVGDRVTITCEAHSQISNYLAWYQQKPGKAP |
| | KLLIFDAKYRAPEVPSRFSGSGSGPDFTLTISSLQPEDFATYYC |
| | QQYNDFWLTFGQGTKVEIK |
| SEQ ID NO: 16 | human C3a |
| | SVQLTEKRMDKVGKYPKELRKCCEDGMRENPMRFSCQRRTRFIS |
| | LGEACKKVELDCCNYITELRRQHARASHLGLAR |
| SEQ ID NO: 17 | Human Factor B |
| | (wt) with signal peptide |
| | MGSNLSPQLCLMPFILGLLSGGVTTTPWSLARPQGSCSLEGVEI |
| | KGGSFRLLQEGQALEYVCPSGFYPYPVQTRTCRSTGSWSTLKTQ |
| | DQKTVRKAECRAIHCPRPHDFENGEYWPRSPYYNVSDEISFHCY |
| | DGYTLRGSANRTCQVNGRWSGQTAICDNGAGYCSNPGIPIGTRK |
| | VGSQYRLEDSVTYHCSRGLTLRGSQRRTCQEGGSWSGTEPSCQD |
| | SFMYDTPQEVAEAFLSSLTETIEGVDAEDGHGPGEQQKRKIVLD |
| | PSGSMNIYLVLDGSDSIGASNFTGAKKCLVNLIEKVASYGVKPR |
| | YGLVTYATYPKIWVKVSEADSSNADWVTKQLNEINYEDHKLKSG |
| | TNTKKALQAVYSMMSWPDDVPPEGWNRTRHVIILMTDGLHNMGG |
| | DPITVIDEIRDLLYIGKDRKNPREDYLDVYVFGVGPLVNQVNIN |
| | ALASKKDNEQHVFKVKDMENLEDVFYQMIDESQSLSLCGMVWEH |
| | RKGTDYHKQPWQAKISVIRPSKGHESCMGAVVSEYFVLTAAHCF |
| | TVDDKEHSIKVSVGGEKRDLEIEVVLFHPNYNINGKKEAGIPEF |
| | YDYDVALIKLKNKLKYGQTIRPICLPCTEGTTRALRLPPTTTCQ |
| | QQKEELLPAQDIKALFVSEEEKKLTRKEVYIKNGDKKGSCERDA |
| | QYAPGYDKVKDISEVVTPRFLCTGGVSPYADPNTCRGDSGGPLI |
| | VHKRSRFIQVGVISWGVVDVCKNQKRQKQVPAHARDFHINLFQV |
| | LPWLKEKLQDEDLGFL |

### EXAMPLES

The following examples are provided to aid the understanding of the present invention, the true scope of which is set forth in the appended claims. It is understood that modifications can be made in the procedures set forth without departing from the spirit of the invention.

### Example 1

### Stabilization of C3BbB Complex

Wild type C3bBb is a rapidly decaying complex unsuitable for antibody discovery purposes. In order to assess the influence of different mutations within the factor B (FB) subunit on the stability of C3bBb the following mutations were introduced in the FB protein: D279G, K350N and M458I. Recombinant FB with a Hig tag and a Precission site is indicated in SEQ ID No: 01, recombinant FB with a D279G mutation is indicated in SEQ ID NO: 02 and recombinant FB with a D279G, K350N and M458I mutations is indicated in SEQ ID NO: 03.

Assembly and decay of the different described C3bBb variants was assessed by Bio Layer Interferometry (BLI) analysis on a sensor coated with C3b.

Stability Detection with BLI

To analyze stability of the C3bBb complexes having the different Bb subunits, C3b cysteine 1010 was biotinylated by incubating plasma purified C3b (Complement Technology) overnight at 4° C. in presence of 5 molar excess Maleimide-PEG3-biotin (Thermo Fisher Scientific) followed by purification on a Zeba spin column 40K (Thermo Fisher Scientific). This material was used to load streptavidin functionalized BLI sensors (SA sensors; Fortebio) in an Octet RED 384 device (Fortebio) at high density (5 nm BLI signal). C3bBb was assembled by exposing the C3b loaded sensors to different recombinant factor B variants (i.e. wild type SEQ ID NO: 1; D279G variant of SEQ ID NO: 2; D279G K350N M458I triple variant of SEQ ID NO: 3) in presence of 54 nM plasma purified factor D (Complement Technology) in HBS-T (Mg) buffer (150 mM NaCl, 10 mM HEPES, 1 mM MgCl2, 0.005% Tween20. pH 7.4) at 22° C. After allowing the convertase to reach equilibrium for 12 minutes, sensors were transferred to 200 μl HBS-T (Mg) buffer initiating dissociation of the assembled C3bBb. Convertase decay was followed for three hours.

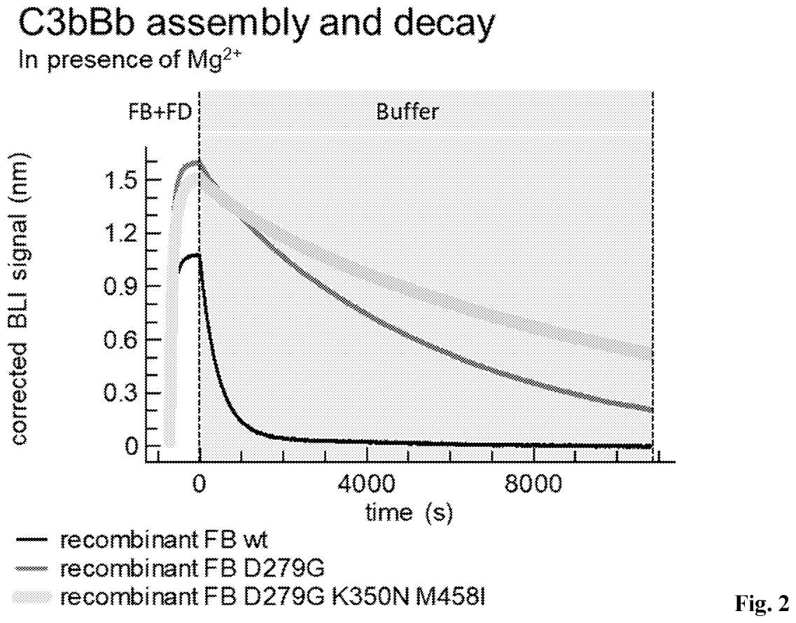

After subtraction of the signal recorded from parallel reference sensors omitting the C3b loading step and subtraction of a control sensor omitting factor B and factor D, data of the dissociation phase was fitted to the double exponential ((A*exp(((−1)*B)*x)))+(C*exp(((−1)*D)*x)) using XLfit (IDBS software), allowing for heterogeneity in the assembled convertase population. Half life (=LN(2)/B) and relative importance of the main species are reported in table 1, BLI trace corrected for nonspecific interactions and signal drift is shown in FIG. 2.

TABLE 1
t½ of C3BbB complex formed using
different Bb proteins as measured by BLI

| | | prevalence of main |
|---|---|---|
| FB used to form C3bBb complex | t ½ | species |
| wt FB | 4 min 46 s | 93% |
| FB (D279G) | 1 h 03 min | 97% |
| FB (D279G, K350N and M458I) | 1 h 59 min | 93% |

The results indicate that a single D279G mutation within the FB protein is suitable to prominently increase the half life of C3BbB, while a triple D279G, K350N and M458I mutation results in further half life increase.

### Assembly in Presence of Nickel Ions

In another experiment, the structural Mg2+ ion coordinated in factor B VWA was substituted for Ni2+ in order to further stabilize the C3 convertase. The setup was identical to the precedent experiment, except that HBS-T (Ni) buffer (150 mM NaCl, 10 mM HEPES, 1 mM NiCl2, 0.005% Tween20. pH 7.4) was used instead of HBS-T (Mg).

Correction of the BLI trace was identical to the experiment in presence of magnesium. Different from the Mg2+ containing C3bBb complexes, only one species was detected during dissociation and a single exponential decay (A*exp(((−1)*B)*x)) was used for analysis.

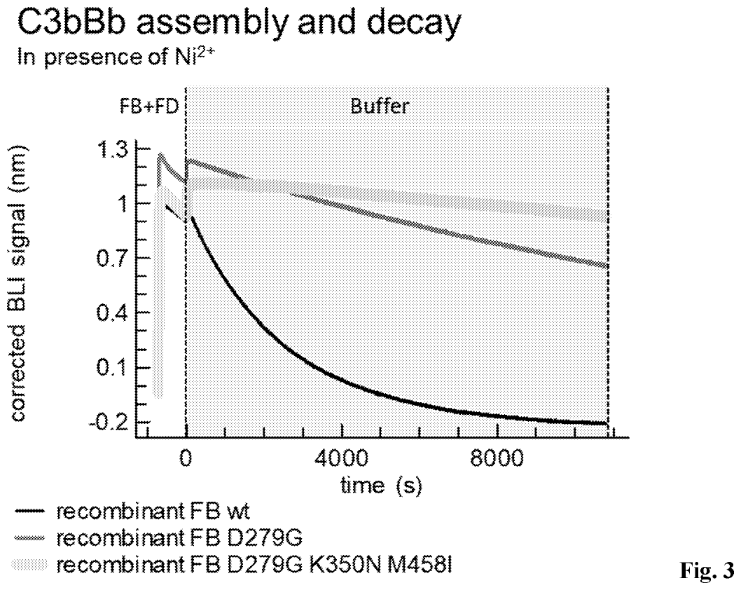

The influence of the addition of Ni2+ ions during assembly of C3BbB using the different Bb proteins, i.e. wild type Bb, single mutated Bb D279G and triple mutated Bb D279G, K350N and M458I for the formation of the C3BbB complex are shown in FIG. 3 and Table 2.

Presence of Ni2+ during formation further stabilizes C3BbB complexes formed with different Bb subunits. Also, the purity of the formed C3bBb is improved.

TABLE 2
t½ of C3BbB complex formed using different Bb
proteins as measured by BLI in presence of Ni²⁺ ions

| | | prevalence of |
|---|---|---|
| FB used to form C3bBb complex | t ½ | main species |
| wt FB | 0 h 29 min | 100% |
| FB (D279G) | 3 h 18 min | 100% |
| FB (D279G, K350N and M458I) | 11 h 18 min | 100% |

While wild type C3BbB complex formed in the presence of magnesium ions had a half life of less than five minutes, stabilization by mutations within the Bb subunit of FB and further stabilizing C3bBb by formation in presence of Ni2+ ions results in a half life of up to 11 hours and 15 minutes (equaling 675 minutes, which equals a more than 135-fold increase in C3bBb half-life).

### Example 2

### Antibody Screening Using Stabilized C3bBb Complex

For screening of antibodies specifically binding to a stabilized C3bBb complex, C3bBb complex was provided by a method according to the invention using a biotinylated C3b for complex assembly, and subsequently, phage library panning was performed using stabilized C3bBb complex as target antigen.

### Assembly and Purification of Stabilized C3bBb Complex for Antibody Discovery

C3b was biotinylated by incubating plasma purified C3b (Complement Technology) for 2 h at room temperature in presence of 0.4 mM EZ-Link HPDP-Biotin (Thermo Fisher Scientific) followed by purification on a NAP-10 Column (Thermo Fisher Scientific). The biotinylated C3b was mixed in a molar ratio of 1:1 with human Factor B (i.e. wild type SEQ ID NO: 1; D279G variant of SEQ ID NO: 2; D279G K350N M458I triple variant of SEQ ID NO: 3), additionally NiSO4 to a final concentration of 5 mM. After 10 minutes of pre incubation at room temperature Factor D (Complement Technology) added in a molar ratio of 1 (C3b): 1 (Factor B): 0.02 (Factor D) and incubated for another 20 min at room temperature in the reaction buffer (20 mM Hepes, 150 mM NaCl, 5 mM NiSO4 pH 7.4). The purification by size exclusion chromatography Superdex200 (Cytavia) followed immediately at room temperature in storage buffer (20 mM Hepes, 150 mM NaCl, 1 mM NiSO4 pH 7.4).

Antibodies specifically binding to the stabilized C3BbB complex were screened as follows:

### Panning

For naïve selection, phage library panning was performed in 4 rounds, wherein the first round was performed with 800 nM of biotinylated C3bBb triple mutant-HPDP (wherein the C3bBb was formed in the presence of nickel ions using FB of SEQ ID NO: 3) pre-immobilized on Dynabeads M-280 Streptavidin (Thermofisher catalog number 11206D). Panning rounds 2-4 were performed with 150 nM of biotinylated target in solution, followed by capture of Fab-on-phage/target complexes on SpeedBead Magnetic Neutravidin Coated Particles (Cytiva, catalog number 78152104010350, round 2 and 4) or Dynabeads M-280 Streptavidin (round 3). In brief, in the first panning round the phage/target/bead complex were washed 2× with HBS-NT+NiSO4 (0.01M HEPES pH7.4; 0.15M NaCl; Cytiva catalog BR-1006-70 supplemented with 0.1% Tween-20 and 1 mM NiSO4) and 1× with HBS-N+NiSO4 (0.01M HEPES pH7.4; 0.15M NaCl; Cytiva catalog BR-1006-70 supplemented with 1 mM NiSO4). In panning rounds 2, 3 and 4 the phage/target/bead complex was washed 5× with HBS-NT+NiSO4 and 2× with 1×HBS-N+NiSO4. Captured phage clones bearing target-specific Fabs were eluted from the M-280 beads using 100 mM DTT, used for infection of log-phase TG1 E. coli cells, and rescued using M13 K07 helper phage, according to standard protocols.

### Screening

For screening of selection outputs, a polyclonal plasmid miniprep of the respective selection round was prepared from the infected TG1 E. coli cells. Plasmids were reformatted to produce soluble Fab in E. coli supernatents with a T7 tag at the C-terminus of the Fab CHI domain. The ligated polyclonal plasmids encoding T7-tagged Fabs were transformed into TG1 E. coli cells (Zymo Research catalog number T3017), and single colonies were picked into microtiter plates. Soluble Fabs were expressed in microtiter plates and supernatants were clarified by centrifugation.

A multitude of antibodies was identified during the screening process and the following antibodies were further analyzed:

TABLE 3
amino acid sequences of identified C3bBb antibodies

| | VH (SEQ ID NO:) | VL (SEQ ID NO:) |
|---|---|---|
| C3bBb.P045.072 | 6 | 7 |
| C3bBb.P048.061 | 8 | 9 |
| C3bBb.P048.127 | 10 | 11 |
| C3bBb.P048.241 | 12 | 13 |
| C3bBb.P055.015 | 14 | 15 |

### Antigen Binding

Antigen binding of the generated Fab fragments was done using an ELISA assay assessing binding towards a C3bBb comprising the Bb subunit of the FB protein of SEQ ID NO: 3 (i.e. the triple mutant), a C3bBb comprising the Bb subunit of the FB protein of SEQ ID NO: 2 (i.e. the single mutant) as well as to C3 and its subunits and FB and its subunits as controls.

### C3bBb-ELISA

Buffer: 50 mM Hepes pH 7.4 containing 150 mM NaCl, 0.5% BSA and 0.1% Tween20 was used as diluent for the ELISA. PBS containing 0.1% Tween20 was used as a washing buffer.

Method: 20 μl of a mixture of 500 ng/ml stabilized human C3bBb-Cys-HPDP-Biotin (inhouse preparation), 1:3000 mouse anti human kappa HRP (Southern Biotech 9230-05) and 1:2000 Mouse IgG anti T7 Tag HRP (Novagen 69048-3) was added to a Microcoat Streptavidine coated 384 well Microplate (Microcoat 11974998001). 25 μl sample was added to the plate and incubated at room temperature for 1 hour. After incubation plates were washed with 90 μl/well washing buffer using a Biotek EL406 Washer and 30 μl TMB (3,3′,5,5′-3,3,5,5-Tetramethylbenzidine, Roche 11835033001) was added. After a 5 Minute incubation at room temperature the optical density (OD) was measured at 370 nm using an En Vision Multimode Plate Reader (Perkin Elmer).

### C3, C3a, C3b Counterscreening Elisa

Buffer: 50 mM Hepes pH 7.4 with 150 mM NaCl was used as coating buffer. 2% BSA in 50 mM Hepes pH 7.4 containing 150 mM NaCl, 0.5% BSA and 0.1% Tween20 was used as blocking buffer. 50 mM Hepes pH 7.4 containing 150 mM NaCl, 0.5% BSA and 0.1% Tween20 was used as diluent for the ELISA. PBS containing 0.1% Tween20 was used as a washing buffer.

Method: A clear 384 well Nunc MaxiSorp Plate (Nunc, 464718) was coated with 25 μl of a mixture of 5 nM human C3 (Complement technology, A113), 5 nM human C3a (Complement technology, A118) and 2.5 nM human C3b (Complement technology, A114) in coating buffer and incubated at room temperature for 1 hour. After a single wash with 90 μl/well washing buffer on a EL406 Biotek washer, 90 μl blocking buffer was added and incubated for 30 Minutes at room temperature. After washing plates 3 times with 90 μl/well washing buffer on a EL406 Biotek washer 25 μl Sample was added to the plate and incubated at room temperature for 1 hour. After washing the plates as described above 25 μl Detection Mix (1:3000 Mouse anti Human kappa HRP (Southern Biotek 9230-05) and 1:2000 Mouse IgG anti T7 Tag HRP (Novagen 69048-3) was added. After a 1 hour incubation at room temperature plates were washed 6 times with 90 μl/well washing buffer using a Biotek EL406 Washer and 30 μl TMB (3,3′,5,5′-3,3,5,5-Tetramethylbenzidine, Roche 11835033001) was added. After a 5 Minute incubation at room temperature the optical density (OD) was measured at 370 nm using a En Vision Multimode Plate Reader (Perkin Elmer).

### Factor B, Ba, Bb Counterscreening Elisa

### Buffers:

50 mM Hepes pH 7.4 with 150 mM NaCl was used as coating buffer 2% BSA in 50 mM Hepes pH 7.4 containing 150 mM NaCl, 0.5% BSA and 0.1% Tween20 was used as blocking buffer.
50 mM Hepes pH 7.4 containing 150 mM NaCl, 0.5% BSA and 0.1% Tween20 was used as diluent for the ELISA. PBS containing 0.1% Tween20 was used as a washing buffer.

Method: A clear 384 well Nunc MaxiSorp Plate (Nunc, 464718) was coated with 25 μl of a mixture of 10 nM human Factor B (Complement technology, A135), 5 nM human Ba (Complement technology, A154) and 10 nM human Bb (Complement technology, A155) in coating buffer and incubated at room temperature for 1 hour. After a single wash with 90 μl/well washing buffer on a EL406 Biotek washer, 90 μl blocking buffer was added and incubated for 30 Minutes at room temperature. After washing plates 3 times with 90 μl/well washing buffer on a EL406 Biotek washer 25 μl Sample was added to the plate and incubated at room temperature for 1 hour. After washing the plates as described above 25 μl Detection Mix (1:3000 Mouse anti Human kappa HRP (Southern Biotek 9230-05) and 1:2000 Mouse IgG anti T7 Tag HRP (Novagen 69048-3) was added in the sample area. After a 1 hour incubation at room temperature plates were washed 6 times with 90 μl/well washing buffer using a Biotek EL406 Washer and 30 μl TMB (3,3′,5,5′-3,3,5,5-Tetramethylbenzidine, Roche 11835033001) was added. After a 5 Minute incubation at room temperature the optical density (OD) was measured at 370 nm using a En Vision Multimode Plate Reader (Perkin Elmer).

TABLE 4
antigen binding as assessed by ELISA

| | C3bBb | | | | |
|---|---|---|---|---|---|
| | (D279G/ | | | | |
| | 350N/ | C3bBb | C3/C3a/ | | |
| | M458I) | (D279G) | C3b | FB/Ba/Bb | control |
| C3bBb.P045.072 | 2.904 | 0.023 | −0.005 | −0.010 | −0.026 |
| C3bBb.P048.061 | 1.899 | 0.004 | 0.001 | 0.006 | 0.015 |
| C3bBb.P048.127 | 0.187 | 2.423 | −0.006 | 0.002 | −0.010 |
| C3bBb.P048.241 | 2.384 | 2.573 | 0.170 | 0.037 | 0.051 |
| C3bBb.P055.015 | −0.010 | 0.860 | −0.014 | 0.004 | −0.014 |

Analysis of the identified antibodies showed antigen binding of stabilized C3bBb complex to either the stabilized C3bBb complex comprising the Bb subunit of the FB protein of SEQ ID NO: 3 (i.e. the triple mutant) or the stabilized C3bBb complex comprising the Bb subunit of the FB protein of SEQ ID NO: 2 (i.e. the single mutant), but not to their subunits and related components of the alternative pathway.

1. A method for providing antibodies specifically binding to C3bBb complex, comprising:
a) providing a C3bBb complex using recombinant human factor B (FB) comprising a D279G mutation, and
b) screening and selecting an antibody that specifically binds to the C3bBb complex provided in step a).

2. A method of decelerating the decay-dissociation of the C3bBb complex, including providing C3bBb complex using recombinant FB comprising a D279G mutation.

3. A method of providing a stabilized C3bBb complex, comprising contacting human Complement C3 beta chain (C3b) with recombinant human FB in aqueous solution, under subsequent addition of human Factor D (FD), and optionally subsequent purification of C3bBb, characterized in that the recombinant human FB comprises a D279G mutation.

4. The method of one of claims 1 to 3, wherein the recombinant FB further comprises a K350N and/or a M458I mutation.

5. The method of one of claims 1 to 3, wherein the recombinant FB further comprises a K350N and a M458I mutation.

6. The method according to any one of the preceding claims, wherein the C3bBb complex is formed by contacting human Complement C3 beta chain (C3b) with recombinant human FB comprising a D279G mutation in aqueous solution, under subsequent addition of human Factor D (FD) and subsequent purification of C3bBb.

7. The method according to any one of the preceding claims, wherein the C3bBb complex is provided by contacting C3b, FB and FD in aqueous solution comprising magnesium ions, preferably Mg²⁺ ions, more preferably MgCl₂.

8. The method according to any one of claims 1 to 7, wherein the C3bBb complex is provided by contacting C3b, FB and FD in aqueous solution comprising nickel ions, preferably Ni2+ ions, more preferably NiSO4.

9. The method for screening antibodies specifically binding to C3bBb complex, according to claims 1 and 4 to 8, wherein the C3bBb complex provided in step a) is used as a target for screening of an antibody that specifically binds to said C3bBb complex.

10. The method according to claim 9, wherein the screening is performed by phage library panning.

11. A recombinant human Factor B protein comprising a D279G mutation.

12. A recombinant human Factor B protein comprising D279G, K350N and/or M458I mutations.

13. A stabilized C3bBb complex comprising a Bb subunit of a recombinant human FB with a D279G mutation, optionally further comprising a K350N and/or a M458I mutation.

14. A stabilized C3bBb complex formed from a recombinant Factor B precursor, wherein the Factor B precursor is a recombinant human Factor B protein according to claim 11 or 12.

15. The recombinant human Factor B protein according to claim 11 or 12 for use in providing a C3bBb complex.

16. The recombinant human Factor B protein according to claim 11 for use in providing a C3bBb complex for use in screening molecules, particularly antibodies specifically binding to the C3bBb complex.


SEQUENCE LISTING (17 sequences)

SEQ ID NO: 1 - protein, 757 aa, synthetic construct
  REGION 1..757: note = artificial sequence
HHHHHHGSLEVLFQGPGGTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 2 - protein, 757 aa, synthetic construct
  REGION 1..757: note = artificial sequence
HHHHHHGSLEVLFQGPGGTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSGSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 3 - protein, 757 aa, synthetic construct
  REGION 1..757: note = artificial sequence
HHHHHHGSLEVLFQGPGGTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSGSIGASNFTGAKKCLVNLIEKVASYGVKPRYGLVTYATYPKIWVKVSEADSSNADWVTKQLNEINYEDHKLNSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDIENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 4 - protein, 227 aa, synthetic construct
  REGION 1..227: note = artificial sequence
ILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVL

SEQ ID NO: 5 - protein, 1641 aa, synthetic construct
  REGION 1..1641: note = artificial sequence
SPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 6 - protein, 116 aa, synthetic construct
  REGION 1..116: note = artificial sequence
KLQLVESGGGLVKPGGSLRLSCAASGRRFGVQDMSWVRQAPGKGLEWVGSISPKGDHKYLNTKFIGRFTISRDNSKNTLYLQMNSLRAEDTAVYYCARDWGYFDQWGQGTLVTVSS

SEQ ID NO: 7 - protein, 107 aa, synthetic construct
  REGION 1..107: note = artificial sequence
AIQMTQSPSSLSASVGDRVTITCHGSYWLSSEVAWYQQKPGKAPKLLIYDGNGIIGNVPSRFSGSGSHEDYTLTISSLQPEDFATYYCQQYRYHPYTFGHGTKVEIK

SEQ ID NO: 8 - protein, 115 aa, synthetic construct
  REGION 1..115: note = artificial sequence
GQLVESGGGLVKPGGSLRLSCKASGFYWGPGAMSWVRQAPGKGLEWVGSISPKGGSTDINPKIGGRFTISRDNNQDTLYLQMNSLRAEDTAVYYCARDIGFFDTWGQGTLVTVSS

SEQ ID NO: 9 - protein, 107 aa, synthetic construct
  REGION 1..107: note = artificial sequence
AIQMTQSPSSLSASVGDRVTITCQGSWLISNYLAWYQQKPGKAPKLLIFDAKYRAPEVPSRFSGSGSGTDFTLTISSLQPEDFATYYCQQYGGRLLTFGQGTKVEIK

SEQ ID NO: 10 - protein, 115 aa, synthetic construct
  REGION 1..115: note = artificial sequence
GQLVESGGGLVKPGGSLRLSCKASGFYWGPGAMSWVRQAPGKGLEWVGSISPKGGSTEYNTKVYGRFTISRDNNQDTLYLQMNSLRAEDTAVYYCARDIGFFDTWGQGTLVTVSS

SEQ ID NO: 11 - protein, 106 aa, synthetic construct
  REGION 1..106: note = artificial sequence
HQMTQSPSSLSASVGDRVTITCDGEWDISNYLAWYQQKPGKAPKLLIFDAKYRAPEVPSRFSGSGSGRDFTLTISSLQPEDFATYYCQQYNRMYFTFGQGTKVEIK

SEQ ID NO: 12 - protein, 115 aa, synthetic construct
  REGION 1..115: note = artificial sequence
GQLVESGGGLVKPGGSLRLSCKASGFYWGPGAMSWVRQAPGKGLEWVGSISPKGGSAWYNNNFTGRFTISRDNNQDTLYLQMNSLRAEDTAVYYCARDIGFFDTWGQGTLVTVSS

SEQ ID NO: 13 - protein, 108 aa, synthetic construct
  REGION 1..108: note = artificial sequence
HINQMTQSPSSLSASVGDRVTITCQAKWVISNYLAWYQQKPGKAPKLLIFDAKYRAPEVPSRFSGSGSDGTFTLTISSLQPEDFATYYCQQYDWWPYTFGQGTKVEIK

SEQ ID NO: 14 - protein, 115 aa, synthetic construct
  REGION 1..115: note = artificial sequence
GQLVESGGGLVKPGGSLRLSCKASGFYWGPGAMSWVRQAPGKGLEWVGSISPKGGSAEYASKVYGRFTISRDNNQDTLYLQMNSLRAEDTAVYYCARDIGFFDTWGQGTLVTVSS

SEQ ID NO: 15 - protein, 107 aa, synthetic construct
  REGION 1..107: note = artificial sequence
DYQMTQSPSSLSASVGDRVTITCEAHSQISNYLAWYQQKPGKAPKLLIFDAKYRAPEVPSRFSGSGSGPDFTLTISSLQPEDFATYYCQQYNDFWLTFGQGTKVEIK

SEQ ID NO: 16 - protein, 77 aa, synthetic construct
  REGION 1..77: note = artificial sequence
SVQLTEKRMDKVGKYPKELRKCCEDGMRENPMRFSCQRRTRFISLGEACKKVFLDCCNYITELRRQHARASHLGLAR

SEQ ID NO: 17 - protein, 764 aa, synthetic construct
  REGION 1..764: note = artificial sequence
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL